(19) United States
(12) Patent Application Publication
(10) Pub. No.: US 2023/0263446 A1
(43) Pub. Date: Aug. 24, 2023

(54) HEART RATE MONITOR

(71) Applicant: Garmin International, Inc., Olathe, KS (US)

(72) Inventors: Mary S. Elder, Raleigh, NC (US); Amy D. Nicholson, Olathe, KS (US); Juliane M. Henne, Overland Park, KS (US); Shawn R. Harvill, Kansas City, KS (US); Sarah C. Bland, Austin, TX (US); Samantha R. Archer, San Francisco, CA (US)

(21) Appl. No.: 18/148,143

(22) Filed: Dec. 29, 2022

Related U.S. Application Data
(60) Provisional application No. 63/312,864, filed on Feb. 23, 2022.

(51) Int. Cl. A61B 5/256; A61B 5/00

(52) U.S. Cl. CPC A61B 5/256; A61B 5/0022; A61B 5/0006; A61B 2503/10; A63B 24/0062

(57) ABSTRACT

A heart rate monitor system can include a number of electrodes, a heart rate module, and a strap. The number of electrodes are configured to contact a chest of a user and detect electrical signals from a heart of the user. The heart rate module is configured to receive the electrical signals from the number of electrodes and wirelessly transmit heart rate data based on the electrical signals. The strap comprises a non-looping strip of material configured to removably couple to a garment of the user.

### RELATED APPLICATIONS

This application claims priority to U.S. provisional application 63/312,864 filed Feb. 23, 2022, the contents of which are hereby incorporated by reference herein for all purposes.

### BACKGROUND

Athletes often wear heart rate monitor straps to accurately measure heart rate during exercise. These straps are designed to tightly wrap around an athlete's chest and include one or more electrodes that must be held against the athlete's chest during exercise to accurately detect electrical signals from the athlete's heart, from which various metrics may be calculated based on heart rate.

### BRIEF DESCRIPTION OF THE DRAWINGS

The detailed description references the accompanying figures. The use of the same reference numbers in different instances in the description and the figures may indicate similar or identical items. In addition, the proportion and the relative scale of the elements provided in the figures are intended to illustrate various embodiments of the present disclosure and are not to be used in a limiting sense.

FIG. 1A is a front view of a heart rate monitor.

FIG. 1B is an isometric view of a strap and an attachment device.

FIG. 1C illustrates an example of a user using a heart rate monitor.

FIG. 2A is an isometric view of an attachment device.

FIG. 2B is an isometric view of a heart rate monitor.

FIG. 3A is an isometric view of an attachment device.

FIG. 3B is an isometric view of an attachment device.

FIG. 3C is an isometric view of an attachment device.

FIG. 4A is an isometric view of a garment and an attachment device in a closed position.

FIG. 4B is an isometric view of a garment and an attachment device in an open position.

FIG. 5A is an isometric view of an attachment portion of a heart rate module in an open position.

FIG. 5B is an isometric view of an attachment portion of a heart rate module in a closed position.

FIG. 6A is an isometric view of an attachment portion of a heart rate module.

FIG. 6B is an isometric view of a strap and an attachment portion of a heart rate module.

FIG. 7A is a front view of a heart rate monitor including an attachment portion in an open position.

FIG. 7B is a front view of a heart rate monitor including an attachment portion in a closed position.

FIG. 7C illustrates an example of a user using a heart rate monitor.

FIG. 8A is a front view of a heart rate monitor.

FIG. 8B is an isometric view of a heart rate monitor.

FIG. 9A is a side view of an attachment portion of a heart rate module in an open position.

FIG. 9B is a side view of an attachment portion of a heart rate module in a closed position.

FIG. 10A is a side view of a heart rate module and a strap.

FIG. 10B is an isometric view of a heart rate module and a strap.

FIG. 10C is an isometric view of a heart rate module and a strap.

FIG. 10D is an isometric view of a heart rate module and a strap.

FIG. 10E illustrates an example of a user using a heart rate monitor.

FIG. 11A is a front view of a heart rate monitor.

FIG. 11B is an isometric view of a heart rate monitor.

FIG. 11C is an isometric view of a heart rate monitor.

FIG. 11D is an isometric view of a strap and an attachment device in an open position.

FIG. 11E is an isometric view of a strap and an attachment device in a closed position.

FIG. 12 illustrates an example of assembling a heart rate monitor.

FIG. 13 illustrates an example of assembling a heart rate monitor.

FIG. 14 is an isometric view of a heart rate module and a strap.

FIG. 15 is a block hardware diagram of a heart rate module.

FIG. 16 is a block hardware diagram of a heart rate monitor system.

### DETAILED DESCRIPTION

The present disclosure includes a heart rate monitor. The heart rate monitor can comprise a number of electrodes, a heart rate module, and a strap. The number of electrodes can contact the chest of a user and detect electrical signals from the heart of the user. The heart rate module can receive the electrical signals from the number of electrodes and wirelessly transmit heart rate data based on the electrical signals. The strap can include a non-looping strip of material that can removably couple to a garment of the user.

Conventional heart rate monitors are often unisex and are not configured to be worn in combination with other garments. For example, current heart rate monitors must be tightly strapped around a circumference of a user's chest in addition to whatever other clothing is worn around the user's chest. Tightly strapped heart rate monitors around a circumference of a user's chest can interfere with a garment the user is wearing, which could cause discomfort or injury to the user. Further, interference between one or more electrodes and a garment can cause the one or more electrodes to dislodge from the skin of the user, which could produce incomplete or inaccurate heart rate data.

The heart rate monitor disclosed herein is a heart rate monitor that is configured to provide accurate and complete heart rate data while easily and comfortably being worn. In some configurations, the heart rate monitor is configured to work in tandem with a user's existing garments.

In various embodiments, the strap of the heart rate monitor has a length roughly corresponding to a width of a typical user's chest and is adapted to attach to the user's sports bra or other garment in a manner that allows electrodes of the heart rate monitor to be tightly held against the user's skin without requiring the strap to itself wrap around the circumference of the user's torso. For example, the strap may extend across a front of the user's torso without extending across the sides or back of the user's torso, or the strap may extend across a portion of the sides of the user's torso without extending across the back of the user's torso. The fabric or base of the strap can be elastic to allow the length of strap to vary based on the particular size of the user.

In some examples, the heart rate monitor can include attachment devices to removably couple the heart rate monitor to an existing garment. The heart rate monitor can use the existing garment to hold the electrodes against the user's chest. The attachment devices can be, but are not limited to, clips, magnets, magnet clips, adhesives, hook and loop fasteners, and/or silicone.

An attachment portion of a heart rate module can also be used to removably couple the heart rate monitor to an existing garment. For example, a heart rate module can include a clip that snaps around a center portion of a user's garment. In a number of embodiments, the heart rate module can be removably coupled to the strap to provide various advantages, such as allowing the strap to be washed without the heart rate monitor attached thereto and allowing the heart rate module to be utilized with different straps and/or garments.

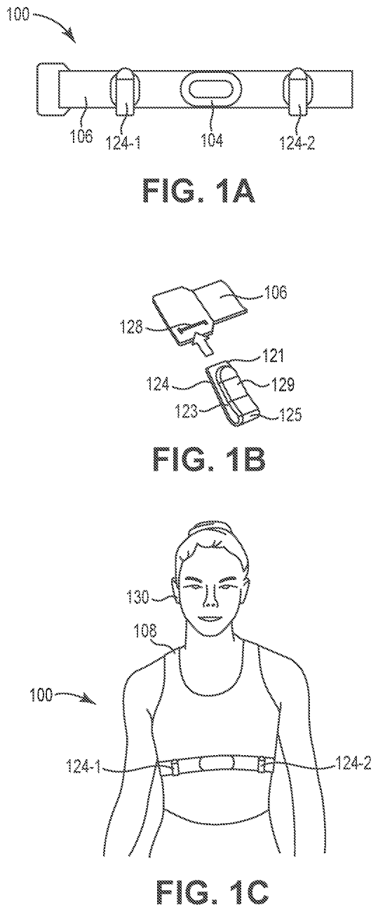

FIG. 1A is a front view of a heart rate monitor 100. Heart rate monitor 100 is operable to record fitness information. The heart rate monitor 100 may be configured in a variety of ways. For instance, heart rate monitor 100 can be configured for use during fitness and/or sporting activities and communicate with a cycle computer, a sport watch, a golf computer, fitness or sporting applications (e.g., apps), a global navigation satellite system (GNSS) receiver used for hiking, and so forth.

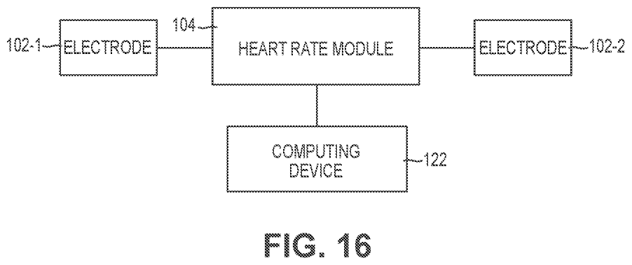

The heart rate monitor 100 includes a strap 106. The strap 106 is configured to receive and/or house (e.g., substantially enclose) various components of the heart rate monitor 100. In some examples, the strap 106 can removably receive a number of electrodes (e.g., electrodes 102-1, 102-2 in FIG. 16) configured to contact a chest of a user and detect electrical signals from a heart of the user. The electrodes can be held to the chest of the user via the strap 106.

The strap 106 can receive and/or house a number of attachment devices 124-1, 124-2 to removably couple the strap 106 to an existing garment. The strap 106 can use an existing garment to hold the electrodes against the user's chest. The attachment devices 124-1, 124-2 can be, but are not limited to, clips, magnets, magnet clips, snaps, adhesives, hook and loop fasteners, and/or silicone. In a number of embodiments, the attachment devices 124-1, 124-2 can include or be made from thermoplastic polyurethane (TPU) or other materials such as other polymers, silicone, metals such as stainless steel, combinations thereof, and the like.

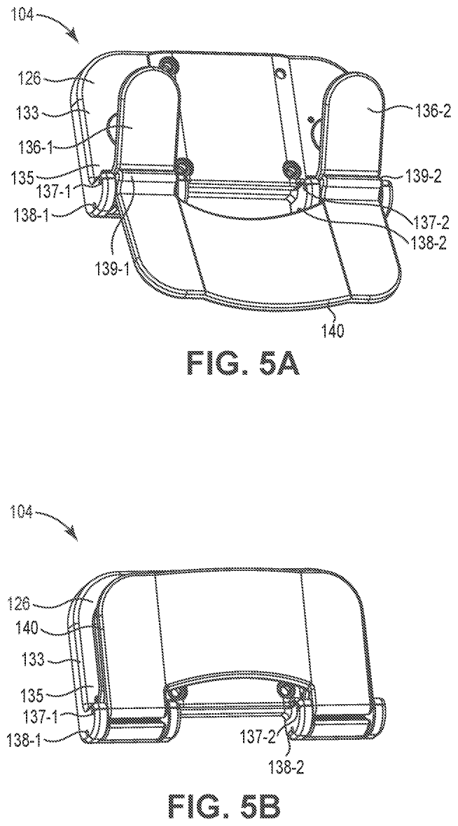

A heart rate module 104 can also be removably received and/or housed by the strap 106. When both the heart rate module 104 and the electrodes are coupled to the strap 106, the electrodes and the heart rate module 104 can be removably coupled to each other. The heart rate module 104 can include an attachment portion (e.g., attachment portion 126 in FIGS. 5A-10D, 12, and 13) used to removably couple the heart rate module 104 to the strap 106 and/or a user's garment. For example, a heart rate module 104 can include an attachment portion with a number of snaps to removably couple the heart rate module 104 to the strap 106. This enables the strap 106 to be washed and the heart rate module 104 to be utilized with different straps and/or garments.

The strap 106 can include a non-looping strip of material that can removably couple to a garment of the user. The material of the strap 106 can be a flexible material to allow the length of the strap 106 to vary based on a user's size. In some examples, the strap 106 can be a flexible TPU wrapped in fabric like polyester or nylon. However, in other examples, the strap 106 can be less flexible (or even inflexible) due to the attachment techniques described herein. In a number of embodiments, the length of the strap 106 can correspond to a width of a typical user's chest. For example, the strap 106 can be 23 centimeters to 41 centimeters in length. The strap 106 can be produced in different sizes (e.g., small, medium, large, etc.). The strap 106 can be described as non-looping because in a number of embodiments, the strap 106 can end before looping around a user's chest. Even when the strap 106 is coupled to the heart rate module 104, the two components do not complete a loop around a user's chest, unlike other traditional heart rate monitor straps, which can form a loop around a user's chest when secured to opposite ends of a heart rate monitor.

FIG. 1B is an isometric view of a portion of a strap 106 and an attachment device 124. The strap 106 may include one or more openings 128. The opening 128 can receive the attachment device 124, as illustrated by the arrow in FIG. 1B, and/or a heart rate module 104 (not specifically illustrated in FIG. 1B). This enables the strap 106 to be washed without the heart rate module attached thereto and the attachment device 124 and/or a heart rate module 104 to be utilized with different straps and/or garments. The strap 106 can receive the attachment device 124 to removably couple the strap 106 to an existing garment. For example, once the attachment device 124 is received by the strap 106, the attachment device 124 can removably couple the strap 106 to a garment.

In a number of embodiments, the attachment device 124 can include a bottom portion 121, a top portion 129, and a hinge portion 125. The opening defined between the bottom portion 121, the hinge portion 125, and the top portion 129 can be referred to as cavity 123.

The attachment device 124 can be formed in such a way and/or formed from such a material that allows the attachment device 124 to be placed into a number of positions. For example, the attachment device 124 can be placed into an open position and/or a closed position when certain forces are applied upon the hinge portion 125 of the attachment device 124. In a steady state, in a number of embodiments, the hinge portion 125 can bias the attachment device 124 to a closed position. The attachment device 124 can be placed in an open position when a force greater than the bias force is applied opposite of or approximately opposite to the bias force of the hinge portion 125. The cavity 123 can receive and/or release a portion of the strap 106 and/or a garment of the user when the attachment device 124 is in an open position. The cavity 123 can removably couple the strap 106 and/or the garment of the user to the attachment device 124 when the attachment device 124 is in the closed position.

FIG. 1C illustrates an example of a user 130 using a heart rate monitor 100. The heart rate monitor 100 can be coupled to a garment 108 the user 130 is wearing via attachment devices 124-1, 124-2. The attachment devices 124-1, 124-2 are illustrated as clips in FIGS. 1A-1C.

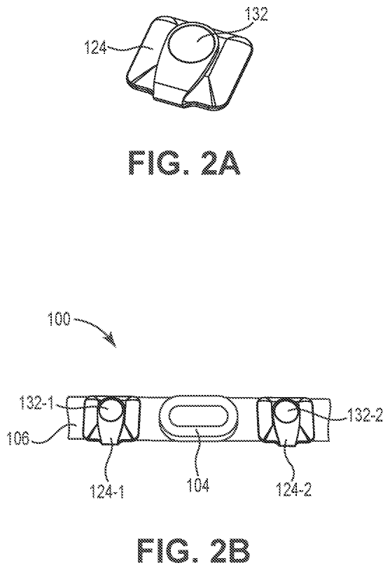

FIG. 2A is an isometric view of an attachment device 124. The attachment device 124 is illustrated as a magnet clip in FIG. 2A. The attachment device 124 can include a magnet 132 to maintain the attachment device 124 in a closed position. In a number of embodiments, magnet 132 can be a permanent magnet embedded into the attachment device 124. Device 124 and magnet 132 can be coupled via a hinge or other physical attachment device and/or be independent elements couplable via magnetic force. In some embodiments, a portion of the attachment device 124 can include and/or be made from a magnetic material. Another portion of the attachment device 124 can include another magnet of a polarity opposite of magnet 132 or be made from a magnetic material that attracts magnet 132 to maintain the attachment device 124 in the closed position.

FIG. 2B is an isometric view of a heart rate monitor 100. The heart rate monitor 100 can include a strap 106. A number of attachment devices 124-1, 124-2 and a heart rate module 104 can be removably coupled to the strap 106. The number of attachment devices 124-1, 124-2 are illustrated as magnet clips including magnets 132-1, 132-2 in FIG. 2B.

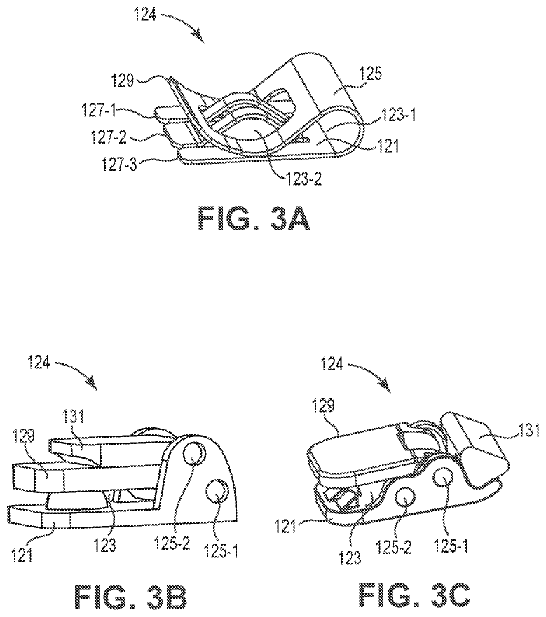

FIG. 3A is an isometric view of an attachment device 124. The attachment device 124 can be a clip, as illustrated in FIG. 3A. A bottom portion 121, a hinge portion 125, a top portion 129, and a number of fingers 127-1, 127-2, 127-3 can be included in the attachment device 124. The opening defined between the bottom portion 121, the hinge portion 125, and the top portion 129 can be referred to as a first cavity 123-1. The opening defined below finger 127-2 can be referred to as a second cavity 123-2.

The attachment device 124 can be formed in such a way and/or formed from such a material that allows the attachment device 124 to be placed into a number of positions. For example, the attachment device 124 can be placed into an open position and/or a closed position when certain forces are applied upon the hinge portion 125 of the attachment device 124. In a steady state, in a number of embodiments, the hinge portion 125 can bias the attachment device 124 to a closed position. The attachment device 124 can be placed in an open position when a force greater than the bias force is applied opposite of or approximately opposite to the bias force of the hinge portion 125.

The first cavity 123-1 can receive and/or release a portion of a garment 108 between the top portion 129 and the bottom portion 121 when the attachment device 124 is in an open position. For example, the garment 108 can travel between finger 127-2 and top portion 129 to reach the first cavity 123-1. A portion of the garment 108 can be enclosed within the first cavity 123-1 and another portion of the garment 108 can be enclosed between the top portion 129 and the number of fingers 127-1, 127-2, 127-3 of the bottom portion 121 when the attachment device 124 is in the closed position.

In some examples, the second cavity 123-2 can receive and/or release a portion of a strap 106. For example, a portion of the strap 106 can be pinned between the fingers 127-1, 127-2, 127-3 by weaving the portion of the strap 106 above fingers 127-1 and 127-3 and below finger 127-2 so that the strap 106 is received by the second cavity 123-2. In other examples, the cavity 123-2 can apply pressure to garment 108 to keep it from slipping instead of pinning or receiving strap 106.

The fingers 127-1, 127-2, 127-3 can be formed in such a way and/or formed from a material having sufficient elasticity to allow the fingers 127-1, 127-2, 127-3 to be placed into a number of positions when certain forces are applied upon them and then to return to a steady state without an opposite force being applied thereto. In a steady state, in a number of embodiments, the fingers 127-1, 127-2, 127-3 can be biased to be coplanar with one another. The fingers 127-1, 127-2, 127-3 can be forced out of alignment when a force greater than the bias force is applied opposite of or approximately opposite to the bias force of at least one of the fingers 127-1, 127-2, 127-3. For example, the fingers 127-1, 127-2, 127-3 can be forced out of alignment when the garment 108 is slid over fingers 127-2 and received in the cavity 123-1.

FIG. 3B is an isometric view of an attachment device 124. The attachment device 124 can be a clip, as illustrated in FIG. 3B. A first hinge portion 125-1, a second hinge portion 125-2, a top portion 129, a bottom portion 121, and a locking portion 131 can be included in the attachment device 124. The opening defined between the bottom portion 121, the first hinge portion 125-1, and the top portion 129 can be referred to as cavity 123.

The attachment device 124 can include the first hinge portion 125-1 that allows the attachment device 124 to be placed into a number of positions. The first hinge portion 125-1 can include a pin secured between openings in opposite sidewalls of the attachment device 124 so as to allow the top portion 129 to rotate about the pin. For example, the attachment device 124 can be placed into an open position and/or a closed position when the top portion 129 is moved. The attachment device 124 can be placed in an open position when the top portion 129 is rotated about the pin of the first hinge portion 125-1 away from the bottom portion 121. A strap 106 and/or garment 108 can be received between the top portion 129 and the bottom portion 121 and into the cavity 123 when the attachment device 124 is in the open position.

The attachment device 124 can be placed in a closed position when the top portion 129 is rotated about the pin of the first hinge portion 125-1 towards the bottom portion 121. A portion of the strap 106 and/or garment 108 can be clamped between the top portion 129 and the bottom portion 121 and enclosed by the cavity 123 when the attachment device 124 is in the closed position. In the closed position, the strap 106 and/or garment 108 can be coupled to the attachment device 124.

In a number of embodiments, the attachment device 124 can include a locking portion 131. The second hinge portion 125-2 can include a pin secured between openings in opposite sidewalls of the attachment device 124 so as to allow the locking portion 131 to rotate about the pin. The locking portion 131 can rotate about the second hinge portion 125-2. The attachment device 124 can be locked in place when the locking portion 131 is parallel to the upper portion 129 and the lower portion 121 in the closed position, as illustrated in FIG. 3B. The locking portion 131 can prevent the top portion 129 and the bottom portion 121 from releasing the strap 106 and/or garment 108. To allow the attachment device 124 to move between the open and closed positions, the locking portion 131 can be rotated away from the top portion 129 and the bottom portion 121.

In a number of embodiments, the bottom portion 121 can be permanently coupled or removably coupled to the strap 106. For example, the bottom portion 121 can be a part of the strap 106 or fastened to the strap 106.

FIG. 3C is an isometric view of an attachment device 124. The attachment device 124 can be a clip, as illustrated in FIG. 3C. A first hinge portion 125-1, a second hinge portion 125-2, a top portion 129, a bottom portion 121, and a locking portion 131 can be included in the attachment device 124. The opening defined between the bottom portion 121, the first hinge portion 125-1, and the top portion 129 can be referred to as cavity 123.

The attachment device 124 can include the first hinge portion 125-1 that allows the attachment device 124 to be placed into a number of positions. For example, the attachment device 124 can be placed into an open position and/or a closed position when the top portion 129 is moved. The first hinge portion 125-1 can include a pin secured between openings in opposite sidewalls of the attachment device 124 so as to allow the top portion 129 to rotate about the pin. The attachment device 124 can be placed in an open position when the top portion 129 is rotated at the first hinge portion 125-1 (e.g., the pin secured between openings in opposite sidewalls of the attachment device 124) away from the bottom portion 121. A strap 106 and/or garment 108 can be received between the top portion 129 and the bottom portion 121 and into the cavity 123 when the attachment device 124 is in the open position.

The attachment device 124 can be placed in a closed position when the top portion 129 is rotated at the first hinge portion 125-1 towards the bottom portion 121. A portion of the strap 106 and/or garment 108 can be clamped between the top portion 129 and the bottom portion 121 and enclosed by the cavity 123 when the attachment device 124 is in the closed position. In the closed position, the strap 106 and/or garment 108 can be coupled to the attachment device 124.

In a number of embodiments, the attachment device 124 can include a locking portion 131. The locking portion 131 can rotate about the second hinge portion 125-2. The attachment device 124 can be locked in place when the locking portion 131 is rotated into contact with the upper portion 129 when the attachment device 124 is in the closed position. The locking portion 131 can prevent the top portion 129 and the bottom portion 121 from releasing the strap 106 and/or garment 108. To allow the attachment device 124 to move between the closed and open positions, the locking portion 131 can be rotated away from the top portion 129.

In a number of embodiments, the bottom portion 121 can be permanently coupled or removably coupled to the strap 106. For example, the bottom portion 121 can be a part of the strap 106 or fastened to the strap 106.

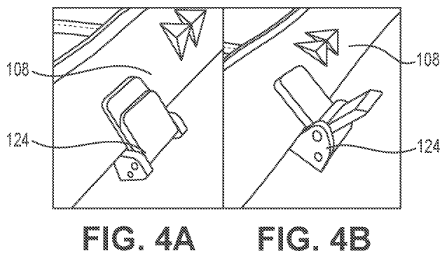

FIG. 4A is an isometric view of a garment 108 and an attachment device 124 in a closed position. The attachment device 124 in FIG. 4A can be a clip corresponding to the attachment device previously described in FIG. 3B.

The attachment device 124 is in a closed position where the garment 108 is clamped between the top portion 129 and the bottom portion 121. In the closed position, the garment 108 can be coupled to the attachment device 124. The attachment device 124 is locked in place by the locking portion 131 to prevent the top portion 129 and the bottom portion 121 from releasing the garment 108.

FIG. 4B is an isometric view of a garment 108 and an attachment device 124 in an open position. In the open position, the garment 108 is not coupled to the attachment device 124 and a portion of the garment 108 is free to enter or leave the attachment device 124.

FIG. 5A is an isometric view of an attachment portion 126 of a heart rate module 104 in an open position and FIG. 5B is an isometric view of the attachment portion 126 in a closed position. The attachment portion 126 can removably couple the heart rate module 104 to a strap 106 and/or garment 108. The attachment portion 126 can include a magnet, a clip, a magnet clip, a snap, an adhesive, a hook and loop fastener, and/or a silicone portion, for example. In some examples, the attachment portion 126 provides an electrical connection between the heart rate module 104 and the strap 106 containing the electrodes 102. Metal contacts, such as snap connectors and sliding interlocks may be utilized to provide electrical conductivity between the module 104 and strap 106, which itself may be electrically connected to one or more of the electrodes 102.

In a number of embodiments, the attachment portion 126 can include a number of hinge portions 138-1, 138-2 that allow the attachment portion 126 to be placed into a number of positions. For example, the attachment portion 126 can be placed into an open position and/or a closed position when a number of top portions 136-1, 136-2 are moved. Each of the hinge portions 138-1, 138-2 can include a pin secured between openings in opposite sidewalls of the attachment portion 126 so as to allow the top portions 136-1, 136-2 to each rotate about a pin. The attachment portion 126 can be placed in an open position when the top portions 136-1, 136-2 are pivoted at the hinge portions 138-1, 138-2 away from the bottom portion 133. The opening defined between the bottom portion 126, the hinge portions 138-1, 138-2, and the top portions 136-1, 136-2 can be referred to as cavity 135. A strap 106 and/or garment 108 can be released from or received between the top portions 136-1, 136-2 and the bottom portion 133 and into the cavity 135 when the attachment portion 126 is in the open position.

The attachment portion 126 can be placed in a closed position when the top portions 136-1, 136-2 are rotated at the hinge portions 138-1, 138-2 towards the bottom portion 133. A portion of the strap 106 and/or garment 108 can be clamped between the top portions 136-1, 136-2 and the bottom portion 133 and enclosed by the cavity 135 when the attachment portion 126 is in the closed position. In the closed position, the strap 106 and/or garment 108 can be coupled to the heart rate module 104.

In a number of embodiments, the attachment portion 126 can include a locking portion 140. The locking portion 140 can rotate about hinge portions 137-1, 137-2. Each of the hinge portions 137-1, 137-2 can include a pin secured between openings in opposite sidewalls of the attachment portion 126 so as to allow the locking portion 140 to rotate about each pin. In some examples, the locking portion 140 can include a number of cam levers 139-1, 139-2. The attachment portion 126 can be locked in the closed position when the cam levers 139-1, 139-2 of the locking portion 140 are rotated into contact with the upper portions 136-1, 136-2, as illustrated in FIG. 5B. The cam levers 139-1, 139-2 of the locking portion 140 can prevent the top portions 136-1, 136-2 and the bottom portion 133 from releasing the strap 106 and/or garment 108. To allow the attachment portion 126 to move between the open and closed positions, the cam levers 139-1, 139-2 of the locking portion 140 can be rotated away from the top portions 136-1, 136-2, as illustrated in FIG. 5A.

In a number of embodiments, the bottom portion 133 can be permanently coupled or removably coupled to the strap 106. For example, the bottom portion 133 can be a part of the strap 106 or fastened to the strap 106.

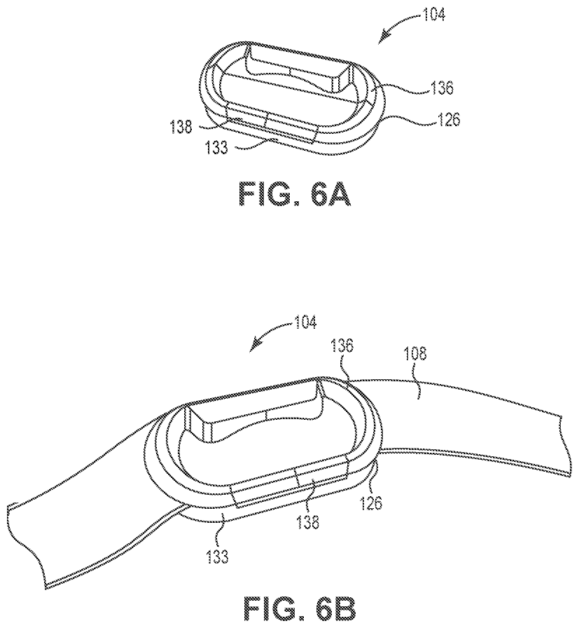

FIG. 6A is an isometric view of an attachment portion 126 of a heart rate module 104 and FIG. 6B is an isometric view of a garment 108 and the attachment portion 126. The attachment portion 126 can be a clip, as illustrated in FIGS. 6A and 6B.

The attachment portion 126 can include a top portion 136, a bottom portion 133, and a hinge portion 138. The top portion 136 and the bottom portion 133 can rotate away from each other to an open position or towards each other to a closed position via the hinge portion 138. A strap 106 and/or a garment 108 can be received by or released from the attachment portion 126 when the attachment portion 126 is in the open position.

FIGS. 6A and 6B illustrate the attachment portion 126 in the closed position. In the closed position, the attachment portion 126 can clamp a strap 106 and/or a garment 108 between the top portion 136 and the bottom portion 133. The attachment portion 126 can removably couple the heart rate module 104 to the garment 108 when the attachment portion 126 is in the closed position, as shown in FIG. 6B.

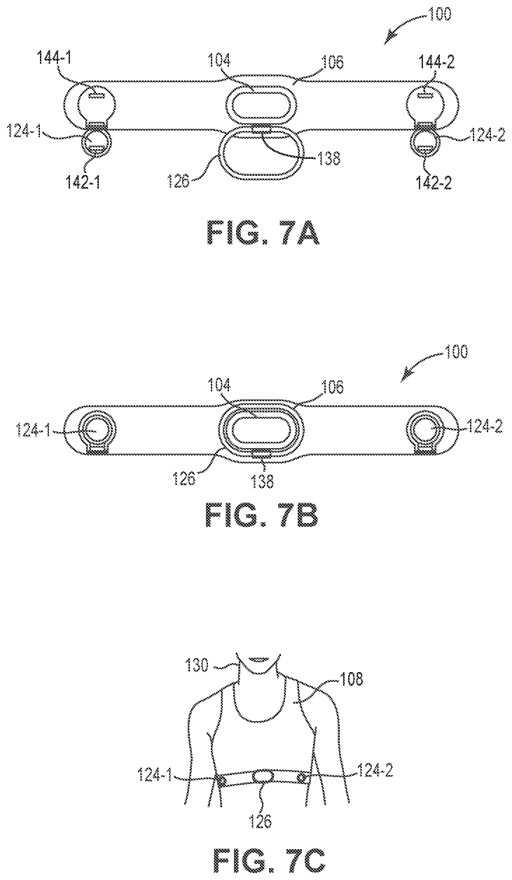

FIG. 7A is a front view of a heart rate monitor 100 including an attachment portion 126 in an open position, FIG. 7B is a front view of the heart rate monitor 100 including the attachment portion 126 in a closed position, and FIG. 7C illustrates a user 130 using the heart rate monitor 100.

The heart rate monitor 100 includes a strap 106. The strap 106 is configured to receive various components of the heart rate monitor 100. In some examples, the strap 106 can house a number of electrodes (e.g., electrodes 102-1, 102-2 in FIG. 16) configured to contact the chest of the user 130 and detect electrical signals from the heart of the user 130. The electrodes can be held to the chest of the user 130 via attachment of the strap 106 the user's garment.

The strap 106 can receive and/or house a number of attachment devices 124-1, 124-2 to removably couple the strap 106 to an existing garment 108. The strap 106 can use an existing garment 108 to hold the electrodes against the user's chest. As illustrated in FIG. 7C, the existing garment 108 can be a sports bra. The strap 106 can be held between the user's body and the existing garment 108 via the attachment devices 124-1, 124-2. The attachment devices 124-1, 124-2 can be snaps, as illustrated in FIGS. 7A, 7B, and 7C. The attachment devices 124-1, 124-2 can each include a socket 142-1, 142-2 and a stud 144-1, 144-2. A protruding portion of the stud 144-1, 144-2 can removably couple to a convex portion of the socket 142-1, 142-2 (e.g., via an interference fit between the stud 144-1, 144-2 and the socket 142-1, 142-2). In some embodiments, a portion of a garment 108 can be placed between the socket 142-1, 142-2 and stud 144-1, 144-2 to removably couple the garment 108 to the strap 106.

A heart rate module 104 can also be received and/or housed by the strap 106. The heart rate module 104 can include an attachment portion 126 used to removably couple the heart rate monitor 100 to the strap 106 and/or a user's garment 108. For example, the attachment portion 126 can be clipped to the heart rate module 104 to removably couple the heart rate module 104 to the garment 108. The attachment portion 126 can be a socket and the heart rate module can have an outer surface forming a stud that produces a friction fit between the attachment portion 126 and the heart rate module 104. A portion of the garment 108 can be inserted between the attachment portion 126 and the heart rate module, which can removably couple the strap 106 to the user's garment 108. In some examples, the attachment portion 126 can be rotatably connected to the heart rate module 104 via a hinge portion 138.

The heart rate module 104, the attachment portion 126, and/or the attachment devices 124-1, 124-2 can be built into the strap 106. The strap 106 can include a non-looping strip of material that can be flexible to allow the length of the strap 106 to vary based on a user's size. In a number of embodiments, the length of the strap 106 can correspond to a width of a typical user's chest, as illustrated in FIG. 7C.

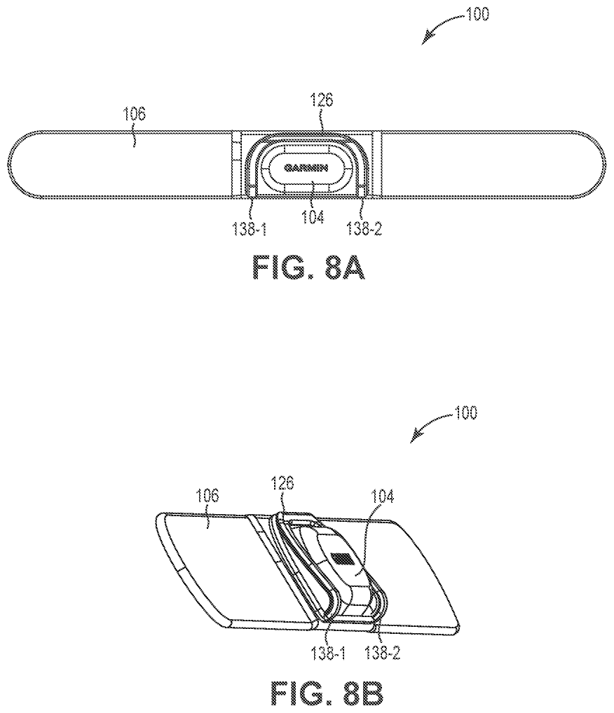

FIG. 8A is a front view of a heart rate monitor 100 and FIG. 8B is an isometric view of the heart rate monitor 100. In a number of embodiments, the heart rate monitor 100 can include strap 106. The strap 106 can include a heart rate module 104 that can be permanently or removably affixed to the strap 106.

The strap 106 can include an attachment portion 126 used to removably couple the strap 106 to a user's garment 108 by inserting the garment 108 between the attachment portion 126 and the heart rate module 104. In some examples, the attachment portion 126 can be rotatably connected to the strap 106 via hinge portions 138-1, 138-2. The hinge portions 138-1, 138-2 can bias the attachment portion 126 toward the strap 106, such as by spring force where the hinge portions 138-1, 138-2 are spring hinges. The attachment portion 126 can be placed into an open position when certain forces are applied upon the hinge portions 138-1, 138-2. For example, the attachment portion 126 can be placed in an open position when a force greater than the bias force is applied opposite of or approximately opposite to the bias force of the hinge portions 138-1, 138-2. The garment 108 can be received between the heart rate module 104 and the attachment portion 126 when the attachment portion 126 is in an open position. In a steady state, in a number of embodiments, the hinge portions 138-1, 138-2 can bias the attachment portion 126 to a closed position and removably couple the strap 106 to the garment 108.

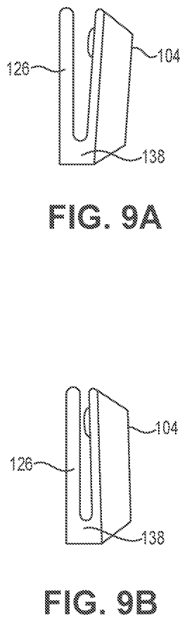

FIG. 9A is a side view of an attachment portion 126 of a heart rate module 104 in an open position and FIG. 9B is a side view of the attachment portion 126 in a closed position.

The heart rate module 104 can include an attachment portion 126 used to removably couple the heart rate module 104 to a user's garment 108 by inserting the garment 108 between the attachment portion 126 and the heart rate module 104. In some examples, the attachment portion 126 can be rotatably coupled to the heart rate module 104 and/or garment via a hinge portion 138. The attachment portion 126 can be placed into an open position, as illustrated in FIG. 9A, when the heart rate module 104 is rotated away from the attachment portion 126 by overcoming the bias force from the hinge portion 138. The garment 108 can be received between the heart rate module 104 and the attachment portion 126 in the open position. In a steady state, in a number of embodiments, the hinge portion 138 can bias the heart rate module 104 and the attachment portion 126 towards each other to a closed position, as illustrated in FIG. 9B, and removably couple the heart rate module 104 to the garment 108. In some examples, a strap 106 can be coupled to the attachment portion 126.

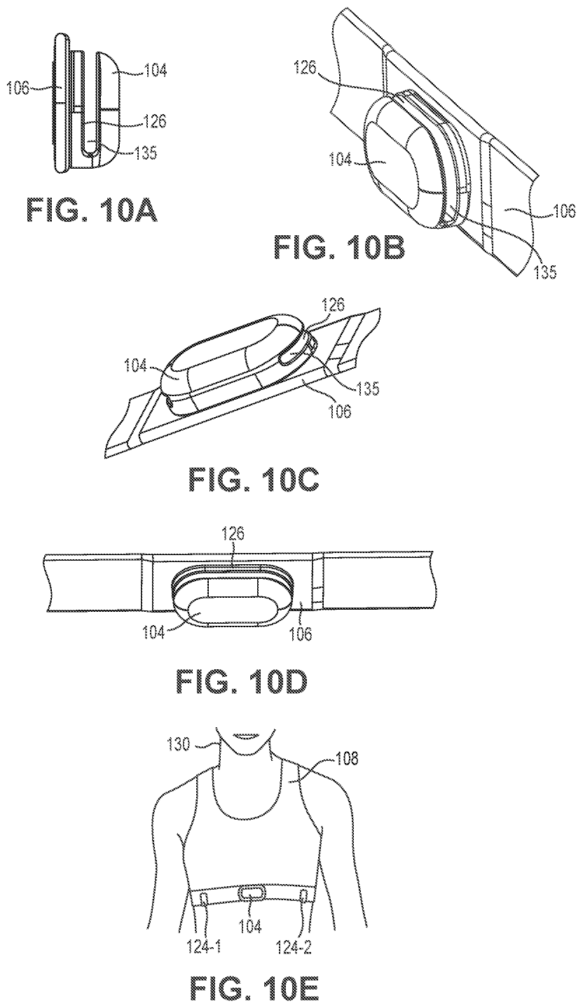

FIG. 10A is a side view of a heart rate module 104 and a strap 106, FIG. 10B is an isometric view of the heart rate module 104 and the strap 106, FIG. 10C is an isometric view of the heart rate module 104 and the strap 106, FIG. 10D is an isometric view of the heart rate module 104 and the strap 106, and FIG. 10E illustrates a user using a heart rate monitor 100 including the heart rate module 104.

The strap 106 can include a heart rate module 104 that can be permanently or removably affixed to the strap 106. The heart rate module 104 can include an attachment portion 126 used to removably couple the heart rate module 104 to a user's garment 108 by inserting the garment 108 into a cavity 135 between the attachment portion 126 and the heart rate module 104.

The strap 106 can receive and/or house a number of attachment devices 124-1, 124-2 to removably couple the strap 106 to the garment 108 of the user 130. As illustrated in FIG. 10E, the existing garment 108 can be a sports bra. The strap 106 can be held between the user's body and the existing garment 108 via the attachment devices 124-1, 124-2.

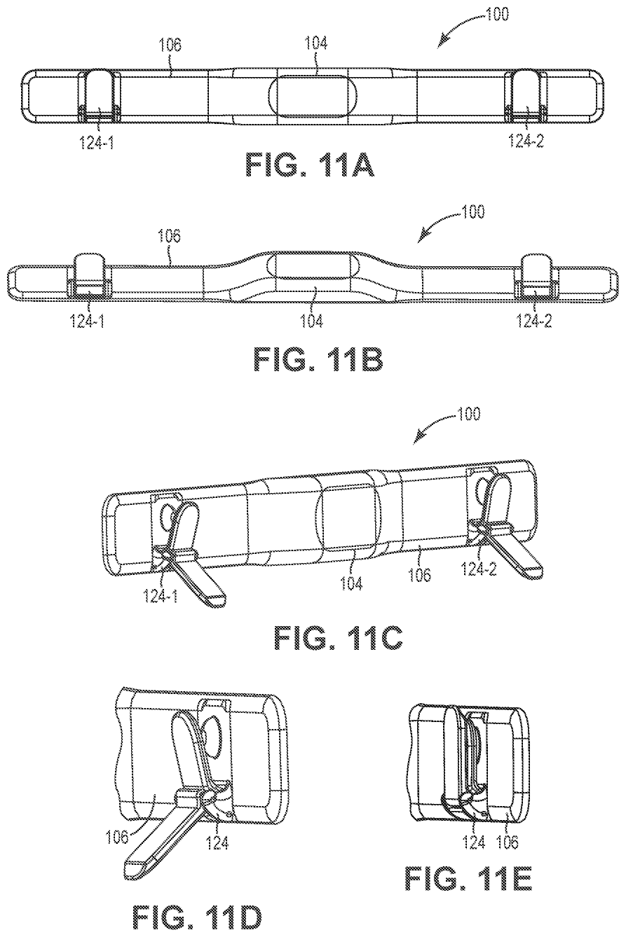

FIG. 11A is a front view of a heart rate monitor 100, FIGS. 11B and 11C are isometric views of the heart rate monitor 100, FIG. 11D is an isometric view of a strap 106 and an attachment device 124 in an open position, and FIG. 11E is an isometric view of the strap 106 and the attachment device 124 in a closed position.

The heart rate monitor 100 can include the strap 106. The strap 106 can include a built-in heart rate module 104, as illustrated in FIGS. 11A, 11B, and 11C. The strap 106 can further include built-in attachment devices 124-1, 124-2 to couple the heart rate monitor 100 to a garment 108.

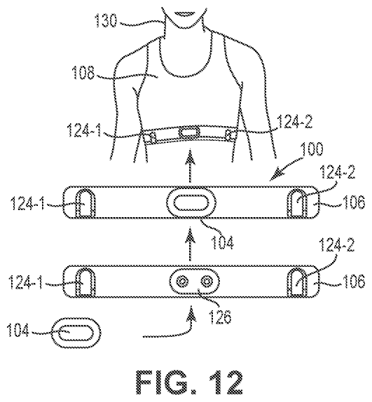

FIG. 12 illustrates an example of assembling a heart rate monitor 100. Starting from the bottom of the illustration in FIG. 12, a heart rate module 104 can be attached to a strap 106 via an attachment portion 126. In some examples, attachment portion 126 attaches strap 106 to garment 108. In other examples, attachment portion 126 attaches heart rate module 104 to strap 106. And, in some examples, attachment portion 126 can attach strap 106 to garment 108 and attach heart rate module 104 to strap 106. For example, the heart rate module 104 can be snapped to the strap 106 when the attachment portion 126 includes a number of socket snaps. The heart rate module 104 can include a number of studs, which can be received by the number of sockets of the attachment portion 126.

Once the heart rate module 104 is coupled to the attachment portion 126, the user 130 can place the attachment devices 124-1 and 124-2 into open positions. Then the heart rate monitor 100 can be inserted between the user 130 and the garment 108. The user 130 can then place the attachment devices 124-1, 124-2 into closed positions enclosing portions of the garment 108 between the strap 106 and the attachment devices 124-1 and 124-2 to removably couple the heart rate monitor 100 to the garment 108 of the user 130.

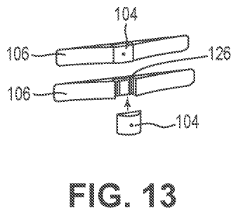

FIG. 13 illustrates an example of assembling a heart rate monitor 100. Starting from the bottom of the illustration in FIG. 13, a heart rate module 104 of the heart rate monitor 100 can be attached to a strap 106 of the heart rate monitor 100 via an attachment portion 126. The attachment portion 126 can include raised portions, for example, rails. The heart rate module 104 can include openings capable of receiving the raised portions of the attachment portion 126 when the heart rate module 104 is slid over the attachment portion 126 to attach the heart rate module 104 to the strap 106 to assemble the heart rate monitor 100.

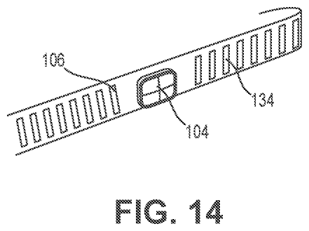

FIG. 14 is an isometric view of a heart rate module 104 and a strap 106. The strap 106 can include features 134 to keep the strap 106 in place on the user's body. These features can include profiles and/or materials that prevent the strap 106 from moving away from the user's body and/or around on the user's body. For example, the strap 106 or a portion thereof can be made out of silicone or silicone can be coupled to portions of the strap 106. As illustrated in FIG. 14, the strap 106 can include a number of features 134 across the horizontal strap 106. The geometry of the features 134 can vary and they can be positioned vertically, horizontally, or in other geometric patterns (e.g., dots) across the strap 106. The features 134 can be a number of silicone portions that enable the strap 106 to grip to the body of the user by providing a greater coefficient of friction between the features 134 and the user's body than between the material forming the strap 106 and the user's body. In some configurations, features 134 can increase friction between the garment 108 and strap 106. Features 134 can generate friction on both the skin side and garment side of the strap 106 to help keep the strap in position.

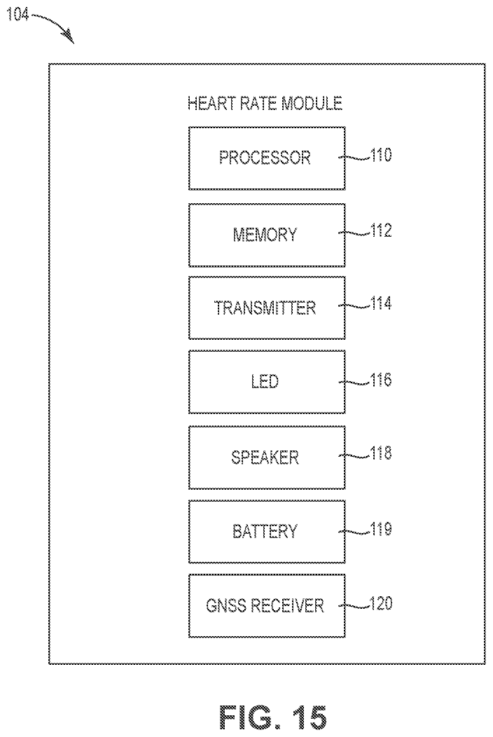

FIG. 15 is a block hardware diagram of a heart rate module 104. The heart rate module 104 can include a processor 110, a memory 112, a transmitter 114, a light emitting diode (LED) 116, a speaker 118, a battery 119, and/or a GNSS receiver 120. The heart rate module 104 may additionally include an accelerometer, a barometric pressure sensor, and/or other sensors and biometric devices to provide functionality to the user.

The processor 110 provides processing functionality for the heart rate module 104 and can include any number of processors, micro-controllers, circuitry, field programmable gate array (FPGA) or other processing systems, and resident or external memory for storing data, executable code, and other information. The processor 110 can execute one or more software programs embodied in a non-transitory computer readable medium (e.g., memory 112) that implement techniques described herein including receiving electrical signals from a number of electrodes (e.g., electrodes 102-1, 102-2 in FIG. 16), wirelessly transmitting heart rate data based on the electrical signals, and/or recording the heart rate of a user 130 based on the electrical signals. The processor 110 is not limited by the materials from which it is formed or the processing mechanisms employed therein and, as such, can be implemented via semiconductor(s) and/or transistors (e.g., using electronic integrated circuit (IC) components), and so forth.

The memory 112 can be a tangible, computer-readable storage medium that provides storage functionality to store various data and/or program code associated with an operation, such as software programs and/or code segments, or other data to instruct the processor 110, and possibly other components of the heart rate module 104, to perform the functionality described herein. The memory 112 can store data, such as program instructions for operating the heart rate module 104 including its components, and so forth. The memory 112 can also store heart rate data, geographic location data, speed data, pace data, cadence data, distance traveled data, calories burned data, and the like. For instance, in some examples, memory 112 may store such data until it can be transferred to another device for further analysis. That is, heart rate module 104 may be configured to operate as a stand-alone device without requiring pairing or connection to another device.

It should be noted that while a single memory 112 is described, a wide variety of types and combinations of memory (e.g., tangible, non-transitory memory) can be employed. The memory 112 can be integral with the processor 110, can comprise stand-alone memory, or can be a combination of both. Some examples of the memory 112 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In a number of embodiments, the heart rate module 104 and/or the memory 112 can include removable integrated circuit card (ICC) memory, such as memory provided by a subscriber identity module (SIM) card, a universal subscriber identity module (USIM) card, a universal integrated circuit card (UICC), and so on.

The transmitter 114 can receive control signals and/or other communications from, for example, a computing device (e.g., computing device 122 in FIG. 16). The transmitter 114 can be communicatively coupled to a computing device via a wired or wireless connection. Accordingly, the transmitter 114 can be a wireless transmitter configured to transmit data, including heart rate data, to a computing device via Bluetooth, Wi-Fi, ANT, a cellular network, and/or other wireless networks.

The LED 116 can be coupled to the heart rate module 104, such that when turned on, the LED 116 is visible to a user 130. The LED 116 can be turned on and/or emit a particular color to indicate to a user 130 that the heart rate module 104 is on, the heart rate module 104 is coupled to the strap 106, electrodes 102-1, 102-2, and/or heart rate monitor 100, the heart rate module 104 is recording data, the heart rate module 104 is malfunctioning, and/or the battery life of the heart rate module 104.

The speaker 118 can be coupled to the heart rate module 104, such that when turned on, the speaker 118 can be heard by a user 130. The speaker 118 can convey messages to the user 130. For example, the speaker 118 can convey that the heart rate module 104 is on, the heart rate module 104 is coupled to the strap 106, electrodes 102-1, 102-2, and/or heart rate monitor 100, the heart rate module 104 is recording data, the heart rate module 104 is malfunctioning, and/or the battery life of the heart rate module 104. In a number of embodiments, the speaker 118 can output heart rate data, geographic location data, speed data, pace data, cadence data, distance traveled data, calories burned data, and the like to the user 130.

The battery 119 can be, but is not limited to being, located within the heart rate module 104. The battery 119 and/or any other power source can be used to power one or more components of the heart rate module 104 and/or one or more components of the heart rate monitor 100 when the heart rate module 104 is coupled to the one or more components of the heart rate monitor 100. In a number of embodiments, the battery 119 can be a coin cell. In some examples, the battery 119 may be or include a thermoelectric generator to convert heat from the user's body into electricity to power the heart rate module 104. Battery 119 may also include a rechargeable battery.

The heart rate module 104 may include one or more sensors for detecting an orientation, change in orientation, direction, change in direction, position, and/or change in position of the heart rate module 104. For example, the heart rate module 104 may include a position determining component that is configured to detect a position measurement for the heart rate module (e.g., geographic coordinates of at least one reference point on the heart rate module 104. In at least one embodiment, the position determining component is a GNSS receiver 120 (e.g., a global positioning system (GPS) receiver, software defined (e.g., multi-protocol) receiver, or the like). Additionally or alternatively, heart rate module 104 may include one or more accelerometers, gyroscopes, tilt sensors, altitude sensors such as barometric pressure sensors, and/or the like.

FIG. 16 is a block hardware diagram of a heart rate monitor system. The heart rate monitor system can include electrodes 102-1, 102-2, a heart rate module 104, and/or a computing device 122.

The heart rate module 104 can be removably coupled to the electrodes 102-1, 102-2 wirelessly and/or via strap 106. The electrodes 102-1, 102-2 can contact a chest of a user 130. In some examples, the electrodes 102-1, 102-2 can be encased in a polymer, plastic and/or other wearable materials or fibers such as elastane to prevent discomfort to the user 130. The electrodes 102-1, 102-2 can detect and/or transmit electrical signals from a heart of a user 130 to the heart rate module 104. The heart rate module 104 can receive the electrical signals and transmit heart rate data and/or record the heart rate data based on the electrical signals.

The computing device 122 can be a cloud device, a tablet, a laptop, a desktop computer, a bike computer, stationary exercise equipment such as a treadmill, a smartphone, and/or a wearable electronic device such as a smartwatch, for example. The computing device 122 can receive data, including heart rate data, from the heart rate module 104. In a number of embodiments, the computing device 122 can store the data in memory and/or convey the received data to a user 130 via a user interface and/or a speaker. The computer device 122 can additionally or alternatively forward data to a cloud service or another internet-connected device for further use.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

As used herein, “a number of” something can refer to one or more of such things. As will be appreciated, elements shown in the various embodiments herein can be added, exchanged, and/or eliminated so as to provide a number of additional embodiments of the present disclosure.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A heart rate monitor system, comprising:
a number of electrodes configured to:
contact a chest of a user; and
detect electrical signals from a heart of the user;
a heart rate module configured to:
receive the electrical signals from the number of electrodes; and
wirelessly transmit heart rate data based on the electrical signals; and
a strap comprising a non-looping strip of material, wherein the strap is configured to removably couple to a garment of the user.

2. The heart rate monitor system of claim 1, wherein the strap is configured to removably receive the number of electrodes and/or the heart rate module.

3. The heart rate monitor system of claim 1, wherein the strap is configured to removably couple the number of electrodes to the heart rate module.

4. The heart rate monitor system of claim 1, wherein the strap is elastic.

5. The heart rate monitor system of claim 1, wherein the heart rate module comprises a processor, a memory, a wireless transmitter, a light emitting diode (LED), a speaker, a battery, and/or a global navigation satellite system (GNSS) receiver.

6. The heart rate monitor system of claim 1, wherein the heart rate module is configured to transmit the heart rate data to a computing device via a wireless network.

7. The heart rate monitor system of claim 6, wherein the computing device is a wearable device, a smartphone, a laptop, a desktop, and/or a cloud device.

8. The heart rate monitor system of claim 1, wherein the number of electrodes are encased in plastic.

9. A heart rate monitor system, comprising:
a number of electrodes configured to:
contact a chest of a user; and
detect electrical signals from a heart of the user;
a heart rate module configured to:
receive the electrical signals from the number of electrodes; and
record a heart rate of the user based on the electrical signals; and
a strap 23 centimeters to 41 centimeters in length configured to:
couple the number of electrodes to the heart rate module; and
removably couple to a garment of the user.

10. The heart rate monitor system of claim 9, wherein the strap is configured to receive a number of attachment devices.

11. The heart rate monitor system of claim 10, wherein the number of attachment devices are configured to removably couple the strap to the garment of the user.

12. The heart rate monitor system of claim 9, wherein the heart rate module comprises an attachment portion configured to removably couple the heart rate module to the garment of the user.

13. The heart rate monitor system of claim 12, wherein the attachment portion of the heart rate module comprises a magnet, a clip, a magnet clip, a snap, an adhesive, a hook and loop fastener, and/or a silicone portion.

14. The heart rate monitor system of claim 12, wherein the attachment portion comprises a cavity configured to receive a portion of the garment of the user.

15. The heart rate monitor system of claim 12, wherein the attachment portion comprises a hinge portion configured to:
receive or release a portion of the garment of the user in an open position; and
removably couple the heart rate module to the garment of the user in a closed position.

16. A heart rate monitor system, comprising:
a number of electrodes configured to:
contact a chest of a user; and
detect electrical signals from a heart of the user;
a heart rate module configured to:
receive the electrical signals from the number of electrodes; and
record a heart rate of the user based on the electrical signals;
a strap configured to couple the number of electrodes to the heart rate module; and
an attachment device configured to removably couple the strap to a garment of the user.

17. The heart rate monitor system of claim 16, wherein the attachment device comprises a magnet, a clip, a magnet clip, a snap, an adhesive, a hook and loop fastener, and/or a silicone portion.

18. The heart rate monitor system of claim 16, wherein the attachment device comprises a cavity configured to receive a portion of the garment of the user.

19. The heart rate monitor system of claim 16, wherein the attachment device comprises a hinge portion configured to:
receive or release a portion of the garment of the user in an open position; and
removably couple the strap to the garment of the user in a closed position.

20. The heart rate monitor system of claim 16, wherein the attachment device is comprised at least partially of plastic.
